(19) United States
(12) Patent Application Publication
(10) Pub. No.: US 2017/0035778 A1
(43) Pub. Date: Feb. 9, 2017

(54) FORMULATIONS AND USES FOR MICROPARTICLE DELIVERY OF METALLOPORPHYRINS

(71) Applicant: The Board of Trustees of the Leland Stanford Junior University, Stanford, CA (US)

(72) Inventors: David K. Stevenson, Los Altos Hills, CA (US); Jayakumar Rajadas, Cupertino, CA (US); Cecilia Espadas, San Jose, CA (US); Ronald James Wong, San Carlos, CA (US); David Lechuga, San Jose, CA (US); Mohammed Inayathullah Nazir Ahmed, Santa Clara, CA (US)

(21) Appl. No.: 15/227,787

(22) Filed: Aug. 3, 2016

Related U.S. Application Data
(60) Provisional application No. 62/220,794, filed on Sep. 18, 2015.
(60) Provisional application No. 62/201,363, filed on Aug. 5, 2015.

(51) Int. Cl. A61K 31/555; A61K 9/16; A61K 9/00

(52) U.S. Cl. CPC A61K 31/555; A61K 9/0053; A61K 9/1617; A61K 9/1635

(57) ABSTRACT

Formulations and methods of use thereof that relate to biocompatible delivery of stabilized porphyrin complexes are provided. Formulations may include microparticles comprising the porphyrin complex, wherein the porphyrin active agent is admixed or coated with a pharmaceutically acceptable stabilizer.

### RELATED APPLICATIONS

The present application claims priority under 35 U.S.C. §119(e) to U.S. provisional patent applications, U.S. Ser. No. 62/201,363, filed Aug. 5,2015, and U.S. Ser. No. 62/220,794, filed Sep. 18, 2015, both of which are incorporated herein by reference.

### GOVERNMENT SUPPORT

This invention was made with government support under contract TW008781 awarded by the National Institutes of Health. The government has certain rights in the invention.

### BACKGROUND

Metalloporphyrins are structural analogs of heme and their potential use in the management of neonatal hyperbilirubinemia and other conditions has been the subject of considerable research for more than three decades. The pharmacological basis for using this class of compounds to control bilirubin levels is the targeted blockade of bilirubin production through the competitive inhibition of heme oxygenase (HO), the rate-limiting enzyme in the bilirubin production pathway. Ongoing research continues in the pursuit of identifying metalloporphyrins that are safe and effective, by defining therapeutic windows and targeted interventions for the treatment of excessive neonatal hyperbilirubinemia.

HO enzymes exist as constitutive (HO-2) and inducible (HO-1) isoforms. The heme oxygenases are metabolic enzymes that utilize NADPH and oxygen to break apart the heme moiety, thereby liberating biliverdin, carbon monoxide (CO) and iron. Biliverdin (BV) and bilirubin, the substrate and product of biliverdin reductase, respectively, are potent antioxidants. The function of HO-1 in cell homeostasis includes the features of acting as a fundamental ‘sensor’ of cellular stress and direct contributor to limit or prevent tissue damage; participation of the products of HO activity in cellular adaptation to stress. In addition to its role in regulating cellular levels of heme, HO is responsible for the recycling of iron from senescent red blood cells and extrahematopoietic cells, such as liver cells. This amount accounts for the high basal activity of HO within those tissues rich in reticuloendothelial cells, such as the spleen and bone marrow.

Pharmacological manipulation of the HO-1 pathway and its products can be used for conferring protection against a variety of conditions characterized by oxidative stress and inflammation.

### SUMMARY

In certain embodiments, the invention relates to formulations and methods of use thereof that relate to biocompatible delivery of an effective dose of a metal porphyrin complex (also referred to herein as an active agent), for example, a metal mesoporphyrin complex or a metal protoporphyrin complex, provided the complex is not zinc protoporphyrin. In some embodiments, the metal is a neutral or ionic atom of an element selected from iron, tungsten, cobalt, magnesium, palladium, platinum, and chromium. For example, the active agent may be hemin. In certain embodiments, the metal is zinc when the active agent is a metal mesoporphyrin complex. In some embodiments, the metal is a neutral or ionic atom of tin.

In some embodiments, the metal porphyrin complex is a heme, for example, heme A, heme B, heme C, or heme O, preferably heme B.

In some embodiments, the complex is formulated for oral delivery. These formulations provide microparticles of a metal porphyrin complex, for example, a metal mesoporphyrin complex or a metal protoporphyrin complex, provided the complex is not zinc protoporphyrin, wherein the metal porphyrin complex is coated with a pharmaceutically acceptable excipient. In some embodiments, a therapeutic composition is provided, comprising a coated microparticle comprising a pharmaceutically acceptable excipient and a metal porphyrin complex, for example, a metal mesoporphyrin complex or a metal protoporphyrin complex, provided the complex is not zinc protoporphyrin. The therapeutic composition may be formulated for oral administration, including without limitation for administration to neonates and infants, e.g., as a liquid, through a gastric tube, etc.

The microparticles described herein comprise the complex and a pharmaceutically acceptable stabilizer, e.g., where the active agent may be at least about 5% of the total microparticle weight, and preferably not more than about 50% of the total microparticle weight. The stabilizer can protect the active agent from instability at low pH, e.g., the acidic conditions present in the stomach. The stabilizer may also increase the solubility of the active agent in neutral pH, e.g., to increase absorption in the neutral conditions present in the intestine.

Methods of treatment with formulations and compositions described herein are also provided. In some embodiments, a method is provided for treating hepatic injury, such as nonalcoholic steatohepatitis (NASH), with any of the active agents described herein, preferably iron protoporphyrin (FePP), comprising administering a microparticulate formulation of an active agent described herein to an individual in need thereof.

In some embodiments, a method is provided for treating cardiovascular disease, such as myocardial ischemia-reperfusion injury, hypertension, or atherosclerosis, including endothelial dysfunction, inflammation, smooth muscle cell proliferation, and vasodilation; diabetes or obesity; hypoxia or ischemia; corneal inflammation; acute kidney injury; hormone dysregulation or imbalance; an infectious disease; cancer; aging, Parkinson's disease, or Alzheimer's disease; or brain hemorrhage, such as subarachnoid hemorrhage, intracerebral hemorrhage, or stroke, with any of the active agents described herein, such as FePP, comprising administering an effective amount of a microparticulate formulation of an active agent described herein to an individual in need thereof.

In other embodiments, a method is provided for treating a porphyria, such as acute hepatic porphyria, comprising administering an effective amount of a microparticulate formulation of an active agent described herein to an individual in need thereof. For example, the active agent is a tin protoporphyrin a tin mesoporphyrin, an iron protoporphyrin, or an iron mesoporphyrin, preferably a heme, more preferably hemin.

### BRIEF DESCRIPTION OF THE DRAWINGS

FIG. 1 depicts a schematic of the luciferase-catalyzed transformation of luciferin to oxyluciferin and light. The transgenic mouse has the full-length HO-1 promoter, which drives expression of the reporter gene, luciferase.

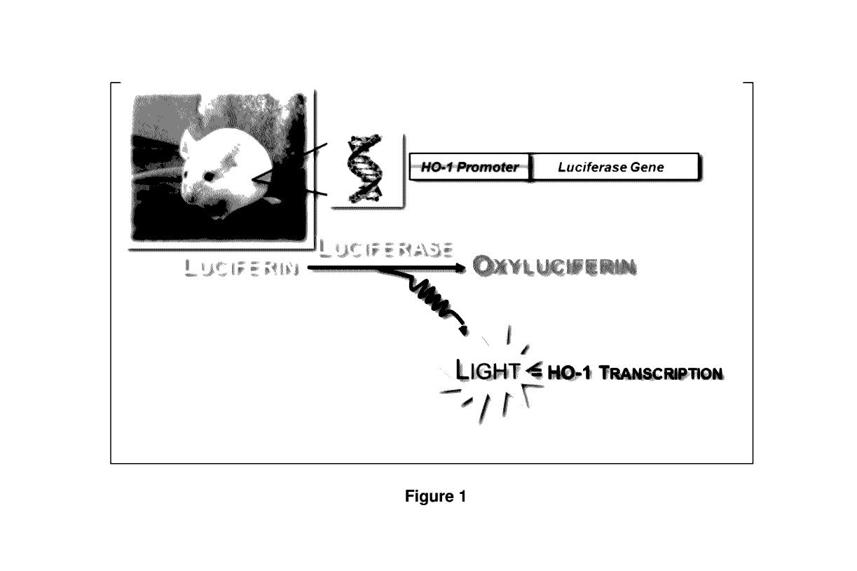

FIG. 2 shows the structure of FePP.

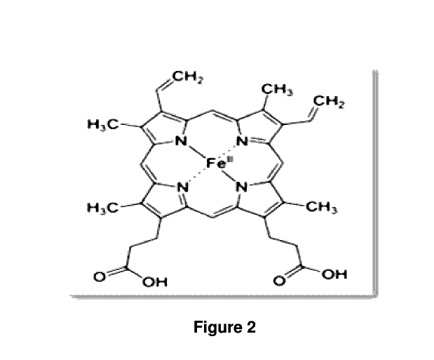

FIG. 3 depicts a timeline for the study of transgenic mice described in Example 2.

FIG. 4 depicts data showing the heme degradation. When VeCO was measured, a significant increase of 1.5- and 2.9-fold compared to baseline levels was observed after treatment with the Heme-Lipid and methemalbumin (MHA) as a source of heme, respectively, peaking at around 2 to 3 hours. In addition, VeCO was significantly higher after MHA administration compared to the heme-lipid.

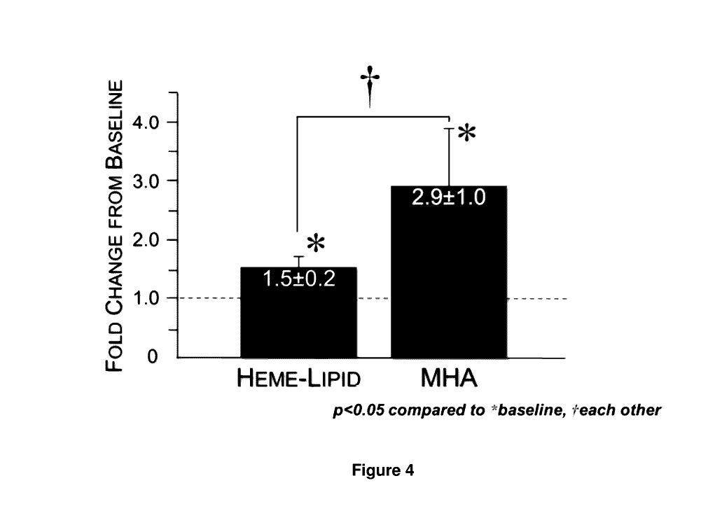

FIG. 5 depicts total HO activity after MHA treatment compared to the Heme-Lipid. All values were significantly higher than control levels.

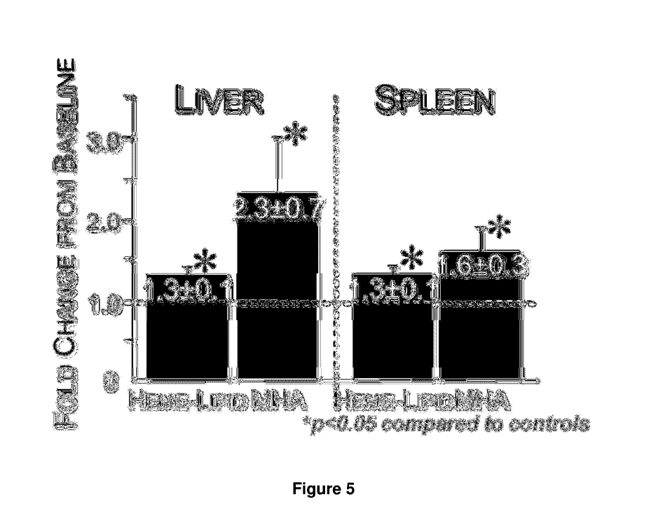

FIG. 6 depicts HO-1 promoter activity. Using BLI, the effect of each preparation on liver and spleen HO-1 transcriptional activity or mRNA levels was investigated. Significant and similar increases over baseline levels in both tissues for both preparation 24 h after administration were observed.

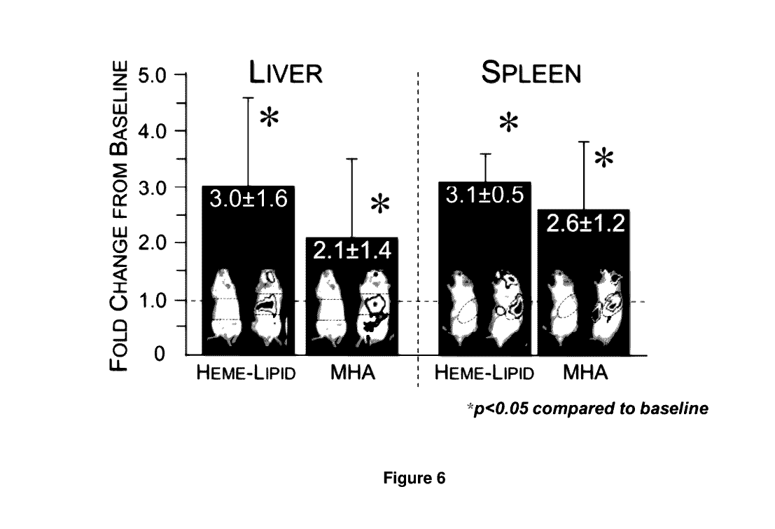

FIG. 7 depicts plasma AST levels. Plasma ALT levels were measured to determine whether the heme-lipid was toxic. No increases in ALT levels following administration of either of the heme preparations were found, compared to controls.

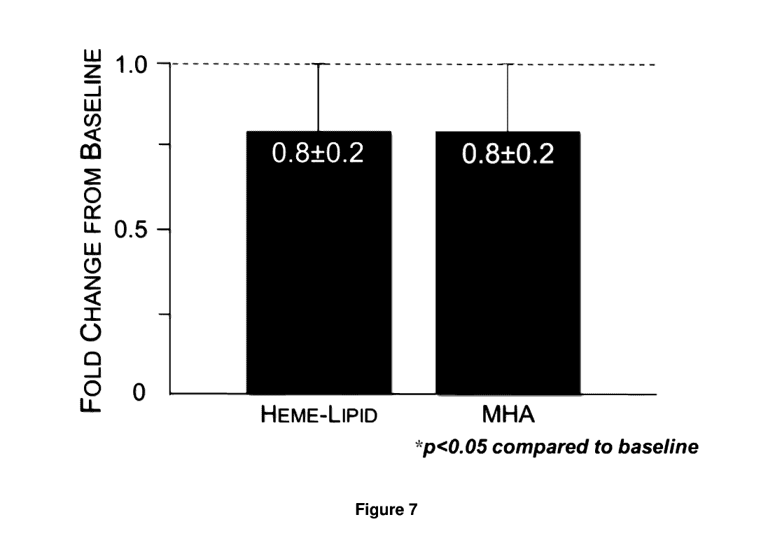

### DETAILED DESCRIPTION OF THE EMBODIMENTS

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges encompassed within the invention, subject to any specifically excluded limit in the stated range.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a microsphere” includes a plurality of such microspheres and reference to “the stent” includes reference to one or more stents and equivalents thereof known to those skilled in the art, and so forth.

Porphyrin. Porphyrins are a group of heterocyclic macrocycle organic compounds, composed of four modified pyrrole subunits interconnected at their a carbon atoms via methine bridges. The parent porphyrin is porphin (or porphine), and substituted porphines are called porphyrins. Porphyrin macrocycles are highly conjugated systems and typically have very intense absorption bands in the visible region. A porphyrin without a metal ion in its cavity is a free base.

Metal porphyrin complexes (metalloporphyrin) comprise an organic porphyrin structure complexed with a metal, e.g. a neutral or ionic atom of an element selected from iron, tungsten, cobalt, magnesium, palladium, platinum, chromium, etc. In complexes other than protoporphyrin, the metal may be zinc, e.g., zinc mesoporphyrin. Included in complexes of interest are metals complexed with protoporphyrin or mesoporphyrin.

Protoporphyrin. Protoporphyrin IX is a tetrapyrrole containing 4 methyl, 2 propionic acid, and 2 vinyl side chains that is a metabolic precursor for hemes, cytochrome c and chlorophyll. Protoporphyrin IX is produced by oxidation of the methylene bridge of protoporphyrinogen by the enzyme protoporphyrinogen oxidase, and is identified as IUPAC 3-[18-(2-carboxyethyl)-8,13-bis(ethenyl)-3,7,12,17-tetramethyl-22,23-dihydroporphyrin-2-yl]propanoic acid.

Mesoporphyrin compounds include any one of the isomeric porphyrins C₃₄H₃₈N₄O₄ produced from protoporphyrin by reducing the vinyl groups to ethyl groups. Included is mesoporphyrin IX, IUPAC designation 3-[18-(2-carboxyethyl)-8,13-diethyl-3,7,12,17-tetramethyl-22,23-dihydroporphyrin-2-yl]propanoic acid, and N-methyl mesoporphyrin, IUPAC designation 3-[18-(2-carboxyethyl)-7,12-diethyl-3,8,13,17,22-pentamethyl-23H-porphyrin-2-yl]propanoic acid.

The terms “treating”, and “treatment” and the like are used herein to generally mean obtaining a desired pharmacological and/or physiological effect. The effect may be therapeutic in terms of a partial or complete cure of a condition, symptom or adverse effect attributed to the condition. The term “treatment” as used herein covers particularly the application of a composition comprising a metal porphyrin complex active agent in microparticle form, including oral administration.

The term “prevent” is art-recognized, and when used in relation to a condition is well understood in the art, and includes administration of a composition which reduces the likelihood of, or delays the onset of, the condition in a subject relative to a subject which does not receive the composition. Thus, prevention of hyperbilirubinemia includes, for example, reducing the likelihood that a subject receiving the composition will experience hyperbilirubinemia relative to a subject that does not receive the composition, and/or delaying the onset of hyperbilirubinemia, on average, in a treated population versus an untreated control population, e.g., by a statistically and/or clinically significant amount. The term “prophylaxis” is used herein to refer to a measure or measures taken for the prevention or partial prevention of a disease or condition.

The term “subject” includes mammals, e.g., cats, dogs, horses, pigs, cows, sheep, rodents, rabbits, squirrels, bears, and primates such as chimpanzees, gorillas, and humans.

The term “pharmaceutically acceptable” as used herein refers to a compound or combination of compounds that will not impair the physiology of the recipient human or animal to the extent that the viability of the recipient is compromised. Preferably, the administered compound or combination of compounds will elicit, at most, a temporary detrimental effect on the health of the recipient human or animal.

The term “carrier” as used herein refers to any pharmaceutically acceptable excipient, diluent, or other dispersant of agents that will allow a therapeutic composition to be administered by the desired route, e.g., by oral administration. A “carrier” as used herein, therefore, includes such excipients and compounds known to one of skill in the art that are pharmaceutically and physiologically acceptable to the recipient human or animal.

The formulations described herein comprise stabilized microparticles of a metalloporphyrin as described above where, relative to the uncoated metalloporphyrin, the microparticles have increased stability in acidic conditions, and/or enhanced solubility at neutral pH. In some embodiments, the microparticles are at least 10% more stable to acidic conditions, at least 20% more stable, at least 30% more stable, at least 40% more stable, at least 50% more stable, at least 75% more stable, and may be at least 2-fold more stable, at least 5-fold, at least 10-fold or more. Stability can be experimentally determined by observing precipitation and degradation in experimental conditions in vitro.

Stabilized microparticle formulations of the invention also confer enhanced absorption at neutral pH, e.g., at a pH greater than 5.5 but less than 8.5, including a pH of greater than about 6.0, greater than about 6.5, greater than about 7.0, and less than about 8.5, less than about 8.0. In some embodiments the microparticles are at least 10% more soluble in neutral pH, at least 20% more soluble, at least 30% more soluble, at least 40% more soluble, at least 50% more soluble, or at least 75% more soluble, and may be at least 2-fold more soluble, at least 5 fold, at least 10-fold or more. Solubility can be experimentally determined by conventional methods.

The microparticle can comprise or consist essentially of an active agent and a stabilizer. In some embodiments, the concentration of the active agent in the microparticle is up to about 5%, up to about 10%, up to about 15%, up to about 20%, up to about 25%, up to about 30%, up to about 35%, up to about 40%, up to about 45%, up to about 50% of the total weight, and the like, and may be from about 5% to about 50%, from about 10% to about 40%, from about 15% to about 35%, from about 20% to about 30% by weight, preferably about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight.

The balance of the microparticle weight is typically provided by stabilizer, i.e., up to about 95%, up to about 90%, up to about 85%, up to about 80%, up to about 75%, up to about 70%, up to about 65%, up to about 60%, up to about 55%, up to about 50%, up to about 45%, up to about 40% of the total weight. In some embodiments, the concentration of stabilizer in the microparticle is preferably about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, or about 70% by weight. The stabilizer confers increased stability at acidic conditions, and allows for increased solubility at neutral pH conditions.

The microparticles may have a controlled size, as appropriate for optimization of drug delivery. Usually the particle will have a diameter of up to about 10 nm, up to about 50 nm, up to about 100 nm, up to about 250 nm, up to about 500 nm, up to about 1 μm, up to about 2.5 μm, up to about 5 μm, and not more than about 10 μm in diameter. In some embodiments, the microparticle size is from about 100 nm to about 5 μm in diameter, for example from about 100 to about 500 nm, from about 500 nm to about 1 μm, and the like. A plurality of microparticles optionally has a defined average size range, which may be substantially homogeneous, where the variability may not be more than 100%, 50%, or 10% of the diameter. Diameters of microparticles may be measured, for example, using scanning electron microscopy (SEM).

Microparticles can be formed by various methods, including, in some embodiments, the methods exemplified herein. Methods of interest may include, without limitation, controlled cation-induced micro-emulsion; and spray drying. Polymeric microparticle fabrication methods can involve polyelectrolyte complex formation, double emulsion/solvent evaporation techniques, emulsion polymerization techniques, and the like. Spray drying is a process that uses jets of dissolved or suspended drug in an aqueous or other fluid phase that is forced through high pressure nozzles to produce a fine mist. Often, a bulking agent will be added to the fluid as well. The aqueous or other liquid contents of the mist evaporate, leaving behind a fine powder. A modification of spray drying, called air nebulization spray drying, uses two wedge-shaped nozzles through which compressed air passes and liquid solutions pass at high velocity. The wedge-shaped nozzle acts as a fluid acceleration zone where the four streams collide at high velocity, producing a shock wave that generates fine droplets. The droplets then descend into a column while being dried into a solid powder by heated air before being collected.

Stabilizers of interest include, without limitation, alginate, chitosan, lecithin, which are naturally occurring mixtures of diglycerides of stearic, palmitic, and oleic acids, linked to the choline ester of phosphoric acid; sodium trimetaphosphate; poloxamers, i.e., nonionic triblock copolymers composed of a central hydrophobic chain of polyoxypropylene (poly(propylene oxide)) flanked by two hydrophilic chains of polyoxyethylene (poly(ethylene oxide)), including various sizes, e.g., Poloxamer 188, poloxamer 407, etc.; cationic lipids, particularly phospholipids; oils, such as coconut oil, etc. Chitosan is a linear polysaccharide composed of randomly distributed β-(1,4) D-glucosamine and N-acetyl-D-glucosamine. Other stabilizers of interest include, for example a protein, such as albumin (for example bovine serum albumin, human serum albumin, etc.), and polyvinylpyrrolidone (PVP) (a water-soluble branched polymer of N-vinylpyrrolidone). PVP useful in the compositions and methods described herein may have a molecular weight of about 10K, or higher, e.g., from about 20K to 50K.

The term “cationic lipids” is intended to encompass molecules that are positively charged at physiological pH, and more particularly, constitutively positively charged molecules, comprising, for example, a quaternary ammonium salt moiety. Cationic lipids used in the methods of the invention typically consist of a hydrophilic polar head group and lipophilic aliphatic chains. See, for example, Farhood et al. (1992) Biochim. Biophys. Acta 1111:239-246; Vigneron et al. (1996) Proc. Natl. Acad. Sci. (USA) 93:9682-9686.

Cationic lipids of interest include, for example, imidazolinium derivatives (WO 95/14380), guanidine derivatives (WO 95/14381), phosphatidyl choline derivatives (WO 95/35301), and piperazine derivatives (WO 95/14651). Examples of cationic lipids that may be used in the present invention include 1,2-distearoyl-sn-glycero-3-phosphocholine (DSPC); 1,2-dipalmitoyl-sn-glycero-3-phosphocholine (DPPC); DOTIM (also called BODAI) (Solodin et al., (1995) Biochem. 34: 13537-13544), DDAB (Rose et al., (1991) BioTechniques 10(4):520-525), DOTMA (U.S. Pat. No. 5,550,289), DOTAP (Eibl and Wooley (1979) Biophys. Chem. 10:261-271), DMRIE (Feigner et al., (1994) J. Biol. Chem. 269(4): 2550-2561), EDMPC (commercially available from Avanti Polar Lipids, Alabaster, Alabama), DCChol (Gau and Huang (1991) Biochem. Biophys. Res. Comm. 179:280-285), DOGS (Behr et al., (1989) Proc. Natl. Acad. Sci. USA, 86:6982-6986), MBOP (also called MeBOP) (WO 95/14651), and those described in WO 97/00241.

In some embodiments, the metalloporphyrin is stabilized in a microparticle formulation with a cationic lipid or lipids. Lipids of interest include any of those listed above, e.g., including DSPC, DPPC, DOTIM, DDAB, DOTMA, DMRIE, EDMPC, DCChol, DOGS, MBOP, etc., which may be used singly or as a cocktail of different lipids, e.g., two lipids at a 10:1, 5:1, 2:1, 1:1, 1:2, 1:5, 1:10, etc. ratio. The lipids can comprise up to about 90% of the microparticle, up to about 85% of the microparticle, up to about 80% of the microparticle, up to about 75% of the microparticle, up to about 70% of the microparticle, up to about 65% of the microparticle, or up to about 50% of the microparticle by weight, where the balance can be the active agent, or can be combined with, for example, EUDRAGIT® L 30 D-55, which is an aqueous dispersion of anionic polymers with methacrylic acid as a functional group, at a concentration of from about 35% to about 75% of the formulation weight. In some embodiments, however, the microparticles are free of EUDRAGIT®. In certain embodiments, the microparticles comprise about 10% to about 25% Metal porphyrin complex by weight, and the balance is a mixture of DSPC and DPPC in the ratios described above.

In some embodiments, the metal porphyrin complex is stabilized in a microparticle formulation with a mixture comprising lecithin, a poloxamer, a neutral oil carrier, e.g., coconut oil, and one or more of alginate, sodium trimetaphosphate and chitosan. In certain embodiments, the microparticles comprise about 5% to about 25% metalloporphyrin, about 10% to about 20% metalloporphyrin by weight. In some such embodiments, the lecithin is present at a concentration of from about 10% to not more than about 40%, from about 20% to not more than about 30% by weight of the microparticle. In some such embodiments, the poloxamer is present at a concentration of from about 10% to not more than about 40%, from about 15% to not more than about 25% by weight of the microparticle. The balance of the formulation comprises, consists essentially, or consists of the neutral oil carrier and the stabilizer.

In some such embodiments, the stabilizer is alginate, which is present at about 3% to about 6%, at about 4% to about 5%, and may be about 4.5% by weight of the microparticle.

In some such embodiments, the stabilizer is chitosan, which is present at about 3% to about 6%, at about 4% to about 5%, and may be about 4.5% to 5% by weight of the microparticle.

In some such embodiments, the stabilizer is sodium trimetaphosphate, which is present at about 3% to about 6%, at about 4% to about 5%, and may be about 4.5% to 5% by weight of the microparticle.

Preferred pharmaceutical compositions are formulated for oral delivery. In some embodiments the microparticles of the invention are provided, e.g., in a unit dose, such as a dry powder for reconstitution immediately prior to administration. Pharmaceutical compositions can include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, or non-toxic, nontherapeutic, non-immunogenic stabilizers, excipients and the like. The compositions can also include additional substances to approximate physiological conditions, such as pH adjusting and buffering agents, toxicity adjusting agents, wetting agents and detergents.

In other embodiments, an oral delivery formulation is provided as a thin film, for example where a dried powder formulation of microparticles is dispersed in a solvent containing a film forming polymer, which can be cast in a thin film and packaged, for example as a unit dose.

Other oral formulations include, without limitation, tablets, lozenges, capsules, sprinkles, sachets, stick-packs, etc. as known in the art and adapted for the microparticles of the invention.

Based on the above, it will be understood by those skilled in the art that a plurality of different treatments and means of administration can be used to treat a single patient.

The total dose per day is preferably administered at least once per day, but may be divided into two or more doses per day. Some patients may benefit from a period of “loading” the patient with a higher dose or more frequent administration over a period of days or weeks, followed by a reduced or maintenance dose.

Specific information regarding formulations which can be used in connection with aerosolized delivery devices are described within Remington's Pharmaceutical Sciences, A. R. Gennaro editor (latest edition) Mack Publishing Company. For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lies within a range of circulating concentrations that include the ED₅₀ with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure. The effective amount of a therapeutic composition to be given to a particular patient will depend on a variety of factors, several of which will be different from patient to patient. A competent clinician will be able to determine an effective amount of a therapeutic agent. The compositions can be administered to the subject in a series of more than one administration.

Those of skill will readily appreciate that dose levels can vary as a function of the specific compound, the severity of the symptoms and the susceptibility of the subject to side effects. Some of the drugs are more potent than others. Preferred dosages for a given agent are readily determinable by those of skill in the art by a variety of means. A preferred means is to measure the physiological potency of a given compound.

### Kits and Packaging

In some embodiments, formulations are provided for use in the methods of the invention. Such formulations may comprise a stabilized microparticle comprising a metalloporphyrin, etc., which can be provided in a packaging suitable for clinical use, including packaging as a lyophilized, sterile powder; packaging of a stable suspension of, for example, microparticles, in carrier; separate packaging of microparticles and carrier suitable for mixing prior to use; and the like. The packaging may be a single unit dose, providing an effective dose of active agent in microparticle form in the manufacture of a medicament, e.g., for improving one or more physiological functions in a patient suffering from NASH, hyperbilirubinemia, ischemia, inflammation, etc.

### Treatment Methods

Methods described herein include the administration, preferably oral administration, of a pharmaceutical composition comprising metalloporphyrin-containing microparticles described herein in a dose effective to inhibit the HO enzyme. Oral administration allows targeted delivery by taking advantage of “first pass effect” resulting in localization to liver and spleen. The metalloporphyrin can also be systemic after oral delivery. The effective dose may vary depending on the age of the individual, the condition being treated, and the like.

Embodiments include methods of treating NASH or the symptoms thereof in an individual. In therapeutic use as agents of the present invention, a microparticle composition comprising a metalloporphyrin may administered at an initial dosage of about 0.1 mg to about 100 mg of active agent/kg BW (IM). In certain embodiments, treatment with the metalloporphyrin is a one-time single dose treatment, in other embodiments, dosing is repeated daily, every 2 days, every 3 days, every 4 days, every 5 days, every 6 days, weekly, every 8 days, every 9 days, every 10 days, every 11 days, every 12 days, every 13 days, every 2 weeks, every 3 weeks, monthly, etc., as need.

Nonalcoholic steatohepatitis (NASH) is a syndrome that develops in patients who are not alcoholic; it causes liver damage that is histologically indistinguishable from alcoholic hepatitis. It develops most often in patients with at least one of the following risk factors: obesity, dyslipidemia, and glucose intolerance. Pathogenesis is poorly understood but seems to be linked to insulin resistance (e.g., as in obesity or metabolic syndrome). Most patients are asymptomatic. Laboratory findings include elevations in aminotransferase levels. Biopsy is required to confirm the diagnosis.

Pathophysiology involves fat accumulation (steatosis), inflammation, and, variably, fibrosis. Steatosis results from hepatic triglyceride accumulation. Possible mechanisms for steatosis include reduced synthesis of very low density lipoprotein (VLDL) and increased hepatic triglyceride synthesis (possibly due to decreased oxidation of fatty acids or increased free fatty acids being delivered to the liver). Inflammation may result from lipid peroxidative damage to cell membranes. These changes can stimulate hepatic stellate cells, resulting in fibrosis. If advanced, NASH can cause cirrhosis and portal hypertension.

The most common laboratory abnormalities are elevations in aminotransferase levels. Unlike in alcoholic liver disease, the ratio of aspartate transaminase/alanine transaminase (AST/ALT) in NASH is usually <1. Alkaline phosphatase and γ-glutamyl transpeptidase (GGT) occasionally increase. Hyperbilirubinemia, prolongation of PT, and hypoalbuminemia are uncommon. For diagnosis, strong evidence that alcohol intake is not excessive is needed, and serologic tests should show absence of hepatitis B and C. Liver biopsy reveals damage similar to that seen in alcoholic hepatitis, usually including large fat droplets (macrovesicular fatty infiltration). Indications for biopsy include unexplained signs of portal hypertension (e.g., splenomegaly, cytopenia) and unexplained elevations in aminotransferase levels that persist for >6 months in a patient with diabetes, obesity, or dyslipidemia. Imaging tests, including ultrasonography, CT, and particularly MRI, may identify hepatic steatosis. However, these tests cannot identify the inflammation typical of NASH and cannot differentiate NASH from other causes of hepatic steatosis.

While the underlying reason for the liver injury that causes NASH is not known, factors may include insulin resistance, release of toxic inflammatory proteins by fat cells (cytokines), and oxidative stress (deterioration of cells) inside liver cells. Elevated levels of serum ferritin and positive liver iron stains are often observed in patients with NASH, and the pathogenesis of liver injury due to iron is also thought to involve oxidative stress. Elevated iron stores as indicated by hyperferritinemia with normal or mildly elevated transferrin saturation and mostly mild hepatic iron deposition are a characteristic finding in subjects with NASH. Excess iron is observed in approximately one third of patients and is commonly referred to as the “dysmetabolic iron overload syndrome”. Clinical evidence suggests that elevated body iron stores aggravate the clinical course with regard to liver-related and extrahepatic disease complications which relates to the fact that excess iron catalyzes the formation of toxic hydroxyl-radicals subsequently resulting in cellular damage. Iron removal improves insulin sensitivity, delays the onset of type 2 diabetes mellitus, improves pathologic liver function tests and likewise ameliorates histology. Several mechanisms contribute to pathologic iron accumulation. These include impaired iron export from hepatocytes and mesenchymal Kupffer cells as a consequence of imbalances in the concentrations of iron regulatory factors, such as hepcidin, cytokines, copper or other dietary factors.

The prevalences of the hemochromatosis protein (HFE) gene mutations associated with hereditary hemochromatosis are increased among subjects with NASH, including the C282Y and H63D mutations.

Hypoxia and Ischemia Ischemia has been found to induce increased expression of HO-1. The interruption of blood supply to an organ can result in a wide variety of metabolic derangements and possible cell necrosis. If the blood supply can be restored (reperfusion) within a certain period of time, cells may recover. However, the process of reperfusion itself can be deleterious, and reperfusion injury has been extensively documented. Reperfusion injury has been defined as the conversion of reversibly injured cells (myocardial, endothelial, and others) to irreversibly injured cells and is mediated by a burst of free radical generation as the previously hypoxic cells are flooded with oxygen. Hypoxia can also aggravate heart disease, resulting in a pressure overload to the right ventricle.

Corneal Inflammation. Processes or agents that produce oxidative tissue injury and inflammation, such as exposure to and absorption of the most energetic wavelengths of direct sunlight, atmospheric oxygen, and a variety of chemical agents known to generate ROS, constantly threaten the corneal epithelium. A common property of many of these agents is their ability to enhance cellular hemeprotein prooxidant systems. The absence of blood vessels in the normal cornea poses a further risk to this tissue because it denies the corneal epithelium access to circulatory plasma-based antioxidant systems. HO in the corneal epithelium may thus participate in protective mechanisms against ocular injury and inflammation. Induction of HO-1 alleviates hypoxic injury-induced ocular surface inflammation and point to a potentially important function for the ocular HO system. Oxidative stress, resulting from various stimuli such as light exposure or free radicals, may be alleviated by enhanced HO activity having an antioxidant effect.

Hyperbilirubinemia. Bilirubin, the product of heme catabolism, is poorly soluble and is thus transported in the circulation tightly, but reversibly, bound to albumin. Bilirubin is then rapidly extracted from the circulation by the liver and bound to cytosolic proteins, which prevent the efflux of the bile pigment back into the circulation. The enzyme UDP-glucuronosyltransferase, an integral membrane protein of the endoplasmic reticulum, conjugates bilirubin with glucuronic acid to form bilirubin-monoglucuronide and bilirubin-diglucuronide, both of which are then excreted in the bile.

Neonatal Jaundice. In newborn humans, the rate of bilirubin production is several-fold greater than that in adults. Peak bilirubin levels in the plasma occur 3 days after birth in full-term infants and are delayed in preterm infants. This increase in plasma bilirubin levels is due, in large part, to the combination of the rapid degradation of fetal hemoglobin in the first few days of life and the immaturity of the hepatic bilirubin-conjugating system, leading to an increase in unconjugated bilirubin. If the levels of unconjugated bilirubin become too high, bilirubin may cross the blood-brain barrier, resulting in bilirubin encephalopathy or kernicterus.

Phototherapy is the current method of choice to lower serum bilirubin levels. Visible light is known to produce photoisomers of bilirubin, which are more water-soluble than bilirubin IX. However, there are increasing concerns regarding the safety of phototherapy, including the possibility of DNA and erythrocyte membrane damage, loss of glucose-6-phosphate dehydrogenase and glutathione reductase activities, and retinal damage. Because phototherapy acts via the skin, only 15% of bilirubin can be photoisomerized at any given time, making phototherapy less efficient for non-neonatal cases of jaundice, such as in children or adults with Crigler-Najjar syndrome. The compositions of the present invention can thus be employed for the management of jaundice by inhibition of HO with metalloporphyrins.

The tin porphyrins, such as tin protoporphyrin (SnPP) and tin mesoporphyrin (SnMP), are potent competitive inhibitors of HO activity. SnPP and SnMP have been shown to control serum bilirubin levels in normal volunteers. There is also substantial clinical experience using SnPP and SnMP to control serum bilirubin levels in patients with hereditary porphyria, liver disease or Crigler-Najjar type I syndrome and in newborns with ABO incompatibility or glucose 6-dehydrogenase deficiency. The results of extensive randomized clinical trials using SnPP and SnMP in particular to control neonatal jaundice indicate that the use of SnMP within 24 h of birth in premature newborns substantially moderates the development of hyperbilirubinemia and markedly reduces the requirement for phototherapy by approximately 75% in inhibitor-treated infants compared with control subjects.

Heme Oxygenase-1 in Diabetes and Obesity. Hyperglycemia, a major cause of kidney disease, is manifest by the development of hypertension and the risk of diabetic neuropathy. Hyperglycemia, defined as elevated levels of serum glucose, produces oxidative stress through elevated levels of ROS, leading to the derangement of cellular physiology. In addition it plays a critical role in the pathogenesis of diabetic complications including cell survival. The impairment of vascular responses to the formation of the super anion radical, O₂⁻, represents the major contributor to vascular injury and the clinical complications of diabetes. The perturbations in heme metabolism manifest via increased levels of HO-1 protein, HO activity, and increased production of CO, iron, and biliverdin/bilirubin.

Heme Oxygenase-1 in Atherosclerosis. Considerable evidence has accumulated to suggest that the HO-1/CO system plays a beneficial role in atherosclerosis. Oxidized LDL, a major determinant in the pathogenesis of atherosclerosis, is a potent inducer of HO-1 in vascular cells. In vascular endothelial cells, vascular smooth muscle cells (VSMCs), and macrophages, HO-1 is markedly up-regulated by oxidized LDL, whereas HO-1 is not increased in vascular endothelial cells or in smooth muscle cells when exposed to native LDL. HO-1 expression is observed throughout the development of atherosclerotic lesions, from the early fatty streaks to advanced complex lesions in human aortic endothelial and smooth muscle cells.

Inflammation and Vascular Injury. Oxidative stress and inflammation are accepted as major factors in the pathogenesis of atherosclerosis. It has been suggested that CO contributes significantly to the anti-inflammatory properties of HO-1. CO inhibits the lipopolysaccharide-mediated expression of proinflammatory cytokines, while simultaneously increasing the expression of the anti-inflammatory cytokine, IL-10, in both endothelial cells and macrophages. Furthermore, HO-1/CO activation down-regulates the inflammatory response by blocking the release of NO from iNOS and expression of the granulocyte-macrophage colony-stimulating factor from macrophages and smooth muscle cells. The activation of both sGC and p38 MAPK has been implicated in the suppression of inflammatory cytokines by HO-1/CO activation. The expression of HO-1 in atherosclerotic plaques and the decrease in experimental atherosclerosis after HO-1 up-regulation further establishes the protective role of HO-1 against atherosclerosis.

Smooth Muscle Cell Proliferation and Vascular Injury. Smooth muscle cell proliferation and monocyte recruitment are essential steps for the development of atherosclerosis. Concomitant with hypoxia-mediated induction of VSMC HO-1, endothelial cell production of endothelin-1, platelet-derived growth factor B, and VEGF was inhibited via a smooth muscle, CO-dependent mechanism. The inhibition of these factors by CO led to a decrease in VSMC proliferation. In addition, smooth muscle cell-derived CO directly decreased VSMC growth by inhibiting E2F-1, a transcription factor that participates in the control of cell cycle progression from the G1 to the S phase.

Heme Oxygenase-1 in Myocardial Ischemia-Reperfusion Injury. Cardioselective overexpression of HO-1 protein exerts a cardioprotective effect after myocardial ischemia-reperfusion in animals, and this effect is probably mediated via the antiapoptotic action of HO-1. Hemin injected before induction of ischemia to upregulate HO-1 decreased left ventricular pressure during ischemia and reperfusion, whereas end-diastolic pressure, coronary perfusion pressure, and coronary resistance increased.

The HO-CO pathway is involved in ischemic vasodilation in the coronary microcirculation. There are numerous possible mechanisms by which the HO-1/HO-2 pathway may improve vascular function. It has been reported that HO-2 activation occurs in ischemic hearts and that inhibition of the HO system inhibits vasodilation during ischemia in the presence of NO and COX inhibitors.

Pharmacological induction of HO-1 significantly reduces infarct size and the incidence of reperfusion arrhythmias after myocardial ischemia-reperfusion. The products of increased HO activity are protective in rodent models of ischemia reperfusion injury, allograft and xenograft survival, intimal hyperplasia after balloon injury, or chronic graft rejection. Up-regulation of HO-1 during heart failure serves to mitigate pathological left ventricular remodeling and reduce myocardial hypertrophy, oxidative stress, and inflammatory activation. Up-regulating HO-1 also has the potential of attenuating cardiac hypertrophy.

Metalloporphyrins, such as heme and heme arginate, are also commonly used to induce HO-1 expression and activity and have been used to normalize blood pressure in animals and human. In the treatment of atherosclerosis, hypertension, and vascular injury in humans, HO-1 has potential of being pharmacologically induced.

Infectious Disease and Heme Oxygenase-1. Infection is associated with a steady and global increase of nonheme iron in the cortex, particularly in neuronal cell bodies of layers II and V, and in capillary endothelial cells. An increase in nonheme iron was associated with the induction of HO-1 in neurons, microglia, and capillary endothelial cells whereas HO-2 levels remained unchanged, suggesting that the nonheme iron increase might be the result of HO-1-mediated heme degradation. Treatment with SnPP (which completely blocked the accumulation of bilirubin detected in HO-1-positive cells) prevented the infection-associated nonheme iron increase.

Cancer and Heme Oxygenase-1. The role of HO-1 in cancer stems from the demonstration that HO-1 is a potent regulator of cell growth and angiogenesis. CO signaling has been established in the promotion of angiogenesis in human microvessel endothelial cells, presumably by increasing the levels of HO derived CO. HO is responsible for prolactin-mediated cell proliferation and angiogenesis in human endothelial cells. In addition, HO-1 has been shown to accelerate tumor angiogenesis in human pancreatic cancer.

Alzheimer's Disease. Neuronal oxidative stress occurs early in the progression of Alzheimer's disease, most notably before the development of pathological hallmarks, such as neurofibrillary tangles and senile plaques. Oxidative stress was correlated with neuropsychological functions, neurofibrillary pathology, and mild cognitive impairment. Therefore, therapeutic efforts aimed to mitigate the deleterious effects of ROS or prevent their formation may prove beneficial. The involvement of the HO pathway is crucial to attenuate the degenerative effect mechanisms operating in Alzheimer's disease.

Parkinson's Disease. Oxidative stress is also a primary pathogenic mechanism of nigral dopaminergic cell death in Parkinson's disease. Oxidative damage (lipid membranes are significantly damaged), Lewy body formation, and decreased mitochondrial complex I activity are the consistent pathological findings. HO-1 is an important cytoplasmic constituent of Lewy bodies, a pathological hallmark of idiopathic Parkinson's disease. Parkinson's and Alzheimer's diseases are associated with elevated iron accumulation relative to the amount of ferritin that is present in the brain. The accumulation of more iron than can be adequately stored in ferritin creates an environment of oxidative stress. The regulation of HO-1 may reflect some fundamental aspect of the pathophysiology of Parkinson's and implicate HO-1 as a useful biological tool for the treatment of this condition.

Intracerebral Hemorrhage. Hemoglobin degradation products produce brain injury after intracerebral hemorrhage. The development of intracerebral hemorrhage-induced hemispheric edema elevates intracranial pressure and can cause death. HO-1 induction is temporally associated with increased tissue heme and is considered a marker for heme-mediated oxidative stress in intracerebral hemorrhage. In survivors, edema-related white matter injury can lead to life-long neurological deficits. Inhibition of HO activity might have potential in the development of new therapies for intracerebral hemorrhage.

Porphyrias. The porphyrias comprise a set of diseases, each representing an individual defect in one of the eight enzymes mediating the pathway of heme synthesis. The diseases are genetically distinct but have in common the overproduction of heme precursors. In the case of the acute (neurologic) porphyrias, the cause of symptoms appears to be overproduction of a neurotoxic precursor. For the cutaneous porphyrias, it is photosensitizing porphyrins. Some types have both acute and cutaneous manifestations.

The porphyrias are grouped according to their predominant manifestation (neurologic or cutaneous) and the tissue that is the main source of porphyrin overproduction (liver or bone marrow). Porphyrias include: 6-aminolevulinic aciduria (ALAD), acute intermittent porphyria (AIP), congenital erythropoietic porphyria (CEP), porphyria cutanea tarda (PCT), hereditary coproporphyria (HCP), variegate porphyria (VP), erythropoietic protoporphyria (EPP), and X-linked protoporphyria (XLP). Acute hepatic porphyrias include: AIP, HCP, VP, and ALAD.

The clinical presentation of acute porphyria manifests as abdominal pain, nausea, and occasionally seizures. Patient populations at increased risk of suffering from a porphyria or presenting with an attack are females between the ages of about 15 and about 45, or individuals of Scandinavian or South African descent.

Currently, treatment of an acute attack initially centers on pain relief and elimination of inducing factors, such as medications, dehydration, or fasting. The only specific treatment is administration of intravenous hemin. An important goal of treatment is preventing progression of the symptoms to a neurological crisis.

In certain embodiments, the invention relates to a method of treating a porphyria or preventing an acute attack of a porphyria in a subject in need thereof, comprising administering to the subject an effective amount of a microparticulate formulation of an active agent described herein, for example, SnPP, SnMP, FePP (such as hemin), or FeMP, preferably the active agent is hemin. Preferably, the porphyria is AIP, HCP, VP, or ALAD. In addition, in some embodiments, the subject is female and about 15 to about 45 years of age; and the composition is administered once a month, for example, about 8 days, about 7 days, about 6 days, about 5 days, about 4 days, about 3 days, about 2 days, or about 1 day prior to menstruation. Preferably, the active agent is administered orally.

In some embodiments, metalloporphyrin is administered in a dosage of from about 0.5 to about 6 mg/kg metalloporphyrin active agent/kg (IM). In some embodiments, metalloporphyrin is administered in a dosage of from about 0.5 mg/kg to about 4 mg/kg, from about 0.5 mg/kg to about 2 mg/kg, from about 0.75 mg/kg to about 1.5 mg/kg, from about 1.5 mg/kg to about 4.5 mg/kg or from about 3.0 mg/kg to about 4.5 mg/kg, including about 1.5 mg/kg, about 3.0 mg/kg and about 4.5 mg/kg. The dosages, however, may be varied depending upon the requirements of the patient, the severity of the condition being treated and the compound being employed. Determination of the proper dosage for a particular situation is within the skill of the art. For example, treatment may be initiated with smaller dosages, which are less than the optimum dose of the compound. Thereafter, the dosage may be increased by small increments until the optimum effect under the circumstance is reached.

Other methods include administering an effective amount of a microparticulate formulation of an active agent described herein to an individual in need thereof, including treatment of conditions comprising, without limitation, NASH, age-related macular degeneration, treatment of infection, cardiovascular disease, such as myocardial ischemia-reperfusion injury, hypertension, or atherosclerosis, including endothelial dysfunction, inflammation, smooth muscle cell proliferation, and vasodilation; diabetes or obesity; hypoxia or ischemia; corneal inflammation; acute kidney injury; hormone dysregulation or imbalance; an infectious disease; cancer; aging, Parkinson's disease, or Alzheimer's disease; or brain hemorrhage, such as subarachnoid hemorrhage, intracerebral hemorrhage, or stroke, with any of the active agents described herein, such as FePP, etc. In various conditions selective modulation of HO is desired. The compositions described herein also find use as contrast enhancing agents for NMR imaging.

Although the present invention has been described in considerable detail with reference to certain preferred embodiments thereof, other versions are possible. Therefore, the spirit and scope of the appended claims should not be limited to the description and the preferred versions contained within this specification.

### EXPERIMENTAL

Bilirubin is formed in the heme catabolic pathway, where heme is degraded by the enzyme, heme oxygenase or HO, to produce equimolar amounts of carbon monoxide, iron, and biliverdin, which is rapidly reduced to bilirubin. In previous work investigating severe hyperbilirubinemia caused by hemolytic diseases, heme bound to albumin or methemalbumin (MHA) was used as a source of heme in acute hemolytic rats, monkeys, and mice. An albumin-free heme preparation was tested for oral bioavailability and subsequent in vivo potency and safety.

### EXAMPLE 1

Formulation contents (w/w): DSPC (45%), DPPC (45%), FePP (10%). Preparation procedure: FePP (10 mg) was dissolved with bubbling oxygen free nitrogen in 10 mL of 1 M ammonium hydroxide solution. DPPC (45 mg) dissolved in 45 mL ethanol was added to it. DSPC (45 mg) dissolved in 45 mL of ethanol was added and mixed well. The solution mixture was spray dried using Buchi-290 mini spray dryer. The set parameters were inlet temperature 55° C.., outlet temperature 50° C.., aspirator at 85%, solution feed pump at 8% and nitrogen flow at 35 mm. The feeding solution container and spray dryer compartments are protected from light during the process. The dry powder is placed under high-vacuum overnight, and then stored −20° C. protected from light.

Preparation of Blank Lipid particles. Formulation contents (w/w): DSPC (50%), DPPC (50%). Preparation procedure: DPPC (50 mg) dissolved in 50 mL ethanol was added to it. DSPC (50 mg) dissolved in 50 mL of ethanol was added and mixed well. The solution mixture was spray dried using Buchi-290 mini spray dryer. The set parameters were inlet temperature 55° C.., outlet temperature 50° C.., aspirator at 75%, solution feed pump at 8% and nitrogen flow at 30 mm. The feeding solution container and spray dryer compartments are protected from light during the process. The dry powder is stored frozen protected from light.

### EXAMPLE 2

Formulation contents (w/w): DSPC (45%), DPPC (45%), FePP (10%). Preparation procedure: FePP (10 mg) was dissolved with bubbling oxygen free nitrogen in 10 mL of 1 M ammonium hydroxide solution. DPPC (45 mg) dissolved in 45 mL ethanol was added to it. DSPC (45 mg) dissolved in 45 mL of ethanol was added and mixed well. The solution mixture was evaporated by Buchi evaporator and made a thin film (117 mbar, 24 h). This was exchanged with water and lyophilized.

Preparation of Blank Lipid particles. Formulation contents (w/w): DSPC (50%), DPPC (50%). Preparation procedure: DPPC (50 mg) dissolved in 50 mL ethanol was added to it. DSPC (50 mg) dissolved in 50 mL of ethanol was added and mixed well. The solution mixture was evaporated by Buchi evaporator and made a thin film (117 mbar, 24 h). This was exchanged with water and lyophilized.

TABLE 1

| DSPC, | DPPC, | Cholesterol, | Hematin | |
|---|---|---|---|---|
| mg | mg | mg | (FePP), mg | # |
| 90 | 0 | 0 | 10 | SP90PP0C0 |
| 65 | 25 | 0 | 10 | SP75PP25C0 |
| 45 | 45 | 0 | 10 | SP5PP5C0 |
| 50 | 40 | 0 | 10 | SP10PP000 |
| 60 | 20 | 20 | 10 | SP60PP40C2 |
| 40 | 30 | 20 | 10 | SP50PP30C2 |
| 30 | 40 | 20 | 10 | SP40PP40C2 |

TABLE 2

| DPPC, | DSPC, | Cholesterol, | Hematin | |
|---|---|---|---|---|
| mg | mg | mg | (FePP), mg | # |
| 90 | 0 | 0 | 10 | PP10SP000 |
| 65 | 25 | 0 | 10 | PP75SP25C0 |
| 40 | 50 | 0 | 10 | PP5SP5C |
| 50 | 40 | 0 | 10 | PP10SP000 |
| 50 | 20 | 20 | 10 | PP60SP40C2 |
| 40 | 30 | 20 | 10 | PP50SP30C2 |
| 30 | 40 | 20 | 10 | PP40SP40C2 |

### Materials and Methods:

Heme-Lipid: This lipid-based formulation contains 10% heme and biodegradable phospholipids (45% DPPC and 45% DSPC), both of which are endogenous phospholipids as well as FDA-approved excipients, and the main constituents of artificial lung surfactant already approved for use in premature newborns. Powder was dissolved in PBS prior to use.

Methemalbumin (MHA): Hemin (chloro[3,7,12,17-tetramethyl-8,13-divinylporphyrin-2,18-dipropanoato(2-)]iron(III)) (Sigma-Aldrich, St Louis, Mo.) was first dissolved in 60 μL of 0.4 M Na₂PO₄. bovine serum albumin (BSA), and 8 mL of dH₂O was then added under constant stirring. 0.1 M HCl was then slowly added until pH was 7.40.

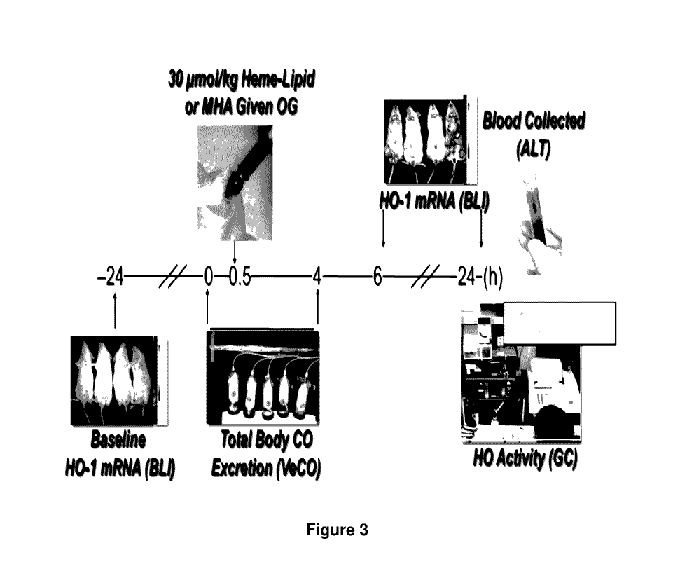

HO-1-luc Transgenic Mouse: This mouse contains a transgene that consists of the full-length HO-1 promoter driving expression of the reporter gene, luciferase (FIG. 3). This model lets us monitor, noninvasively, any changes in HO-1 transcription through proportional changes in luciferase transcription and activity. When luciferin is administered to the transgenic mice, it is rapidly converted to oxy-luciferin by luciferase resulting in the production of light.

HO-1 Transcription (BLI): For these measurements, adult mice were imaged using the IVIS. Bioluminescence was then quantitated at the ventral (for liver) and left lateral (for spleen) regions as total number of photons emitted per second or flux. HO-1 promoter activity was then expressed as fold change from baseline levels.

Heme Degradation (VeCO): Because CO and bilirubin are produced in equal molar amounts during heme degradation, total body CO excretion or VeCO can be used as an index of bilirubin production. For these measurements, adult mice were placed in 50-mL chambers with an air flow of 40 mL/min and monitored for up to 6 h. CO in the chamber outlet air was measured by gas chromatography (GC). VeCO was then expressed as μL CO excreted/h/kg bodyweight and fold change from baseline levels was calculated.

Plasma AST Levels: Blood was collected at sacrifice by intracardiac puncture. Plasma was then separated by centrifugation and sent to the Diagnostics Laboratory (Dept. of Comparative Medicine, Stanford, Calif.) for measurements of plasma AST levels.

HO Activity: Total HO enzyme activity in 20 μL of liver or spleen sonicates was measured by GC. Data were expressed as μmol CO produced/h/mg fresh weight and fold change from control levels were calculated.

Statistics: All comparisons were analyzed using Student's unpaired t-test. Data are expressed as mean±SD. Differences were considered significant when p≦0.05.

Heme Degradation. When VeCO was measured, we saw a significant increase of 1.5- and 2.9-fold compared to baseline levels after treatment with the Heme-Lipid and MHA, respectively, peaking at around 2 to 3 h. In addition, VeCO was significantly higher after MHA administration compared to the heme-lipid. Liver HO activity was higher, but not significantly, after MHA treatment compared to the Heme-Lipid. A similar trend was observed for the spleen. All values were significantly higher than control levels.

HO-1 Promoter Activity. When we compared the effect of each preparation on liver and spleen HO-1 transcriptional activity or mRNA levels using BLI, we observed significant and similar increases over baseline levels in both tissues for both preparation 24 h after administration.

Plasma AST Levels. To see if there was any toxicity related to the Heme-Lipid, we measured plasma ALT levels. We found no increases following administration of either of the heme preparations compared to controls.

In summary, compared to MHA, heme-lipid showed similar potency by significantly: increasing in vivo bilirubin production rates; upregulating HO-1 mRNA in liver and spleen; and inducing HO activity in the liver and spleen. Neither MHA nor Heme-Lipid increased ALT levels.

### EXAMPLE 3

Microparticles are formed by spray drying which ensures a straightforward path for scale-up under a GMP environment which will be required for the GMP manufacturing of a final dosage form. Spray dried powder preparation; formulation components:

metalloporphyrin; 20% w/w

DPPC; 5% w/w

EUDRAGIT® L100-55; 75% w/w

Preparation of 1% solids (w/v) feedstock solution for spray drying: metalloporphyrin is dissolved with sonication in 1 M ammonium hydroxide (constituting 12.5% of total solvent volume). DPPC is added dissolved in ethanol (12.5% of total solvent volume). EUDRAGIT® L100-55 is added dissolved in ethanol (remaining 75% solvent volume). Büchi-290 spray drying conditions:

Inlet temperature: 70° C.

Outlet temperature: 50° C.

Aspirator: 75%

Nitrogen: 30 mm gauge height

Pump: 8%

The feeding solution container and spray dryer compartments are protected from light during the process. The dry powder is stored frozen protected from light.

### EXAMPLE 4

### Spray Dried Powder Preparation with Higher Metalloporphyrin Content

Metalloporphyrin formulation with lower EUDRAGIT® content; formulation components:

metalloporphyrin; 20% w/w

DPPC; 42% w/w

EUDRAGIT® L100-55 (poly(methacrylic acid-co-ethyl acrylate) 1:1, approx. 320,000 g/mol); 38% w/w

Preparation of 1% solids (w/v) feedstock solution for spray drying: metalloporphyrin is dissolved with sonication in 1 M ammonium hydroxide (constituting 12.5% of total solvent volume). DPPC is added dissolved in ethanol (50% of total solvent volume). EUDRAGIT® L100-55 is added dissolved in ethanol (remaining 37.5% solvent volume). Spray drying conditions: same as described above.

### EXAMPLE 5

### Powder Containing Vial or Syringe to be Reconstituted with an Appropriate Diluent to be Orally Delivered

The desired dose of metalloporphyrin may be contained in a small amount of spray-dried powder or less depending on the final metalloporphyrin content and depending on the target subject. Spray-dried powders are usually small in size and tend to be more cohesive than granular powders. A bulking agent can be used to blend the spray-dried powder to facilitate filling into a vial to be then resuspended with an appropriate diluent prior to administration to the test subject. This can be achieved as described below.

Metalloporphyrin -DPPC- EUDRAGIT® spray-dried powder is mixed with D-glucose as bulking agent to obtain a uniform mixture containing the target amount of spray dried powder, calculated based on the amount of metalloporphyrin content and the required dose, with an appropriate amount of D-glucose (ranging in the amount of 10% to 90% w/w) the powder blend can then be filled in a glass or plastic vial or syringe manually or using a filling machine.

Prior to administration, a suspension is then formed by adding the appropriate amount of diluent containing 0.25% (w/v) citrate buffer, pH 4.7, to the target amount of powder containing the required dose. The pH of the diluent is selected to minimize dissolution of the polymer microparticles, but not too low to cause chemical degradation of the metalloporphyrin. The suspension is agitated and ready for administration.

### EXAMPLE 6

### Metal Porphyrin Complex Oral Thin Film Dosage Form

The metalloporphyrin spray-dried powder formulation can be dispersed in a solution of an organic solvent containing a film-forming polymer. The polymer solution containing the suspended spray-dried powder is then cast into a thin film, which is then cut in appropriate size sections, each of the sections containing one dose. The thin film is expected to instantaneously dissolve in the mouth without the need of any extra liquid.

### EXAMPLE 7

### Metal Porphyrin Complex Fast Dissolve Tablet

The metalloporphyrin spray-dried powder formulation can be suspended in diluent containing 0.25% (w/v) citrate buffer, pH 4.7 to which mannitol can be added in an appropriate amount. The suspension is then transferred into tablet-size molds, which then are frozen in a stream of liquid nitrogen. The frozen suspension is then lyophilized and the mold containing the tablets obtained can be sealed with a protective cover. The lyophilized tablets are expected to instantaneously dissolve in the mouth without the need of any extra liquid.

### EXAMPLE 8

### Preparation of Metalloporphyrin/Chitosan Microparticles

Formulation contents (w/w): Coconut oil (40%), Lecithin (30%), metalloporphyrin (5%), Poloxamer-188 (20%) and Chitosan (MW-15000) (5%)

Preparation procedure: Coconut oil (40 mg) and Lecithin (30 mg) are dissolved in 10 mL of ethanol by stirring at room temperature (RT). To this solution, metalloporphyrin (5 mg) is added and heated to 40° C. while stirring to obtain a clear solution. Separately, Poloxamer-188 (20 mg) is dissolved in 8 mL of distilled water and then added to the above ethanol solution while stirring at room temperature. Chitosan-15K (5 mg) is dissolved in 1 mL of 0.01 N aqueous hydrochloric acid and then added to the above mixture solution stirring at room temperature. The solvents are removed from this homogeneous solution under vacuum using a rotary evaporator. The residue is reconstituted with 10 mL of water by sonication for 20 min. The solution is frozen and lyophilized to obtain the final microparticles.

### EXAMPLE 9

### Preparation of Metalloporphyrin/Alginate Microparticles

Formulation contents (w/w): Coconut oil (35%), Lecithin (20%), metalloporphyrin (20%), Poloxamer-188 (20%) and Sodium Alginate (4.5%) Calcium chloride (0.5%)

Preparation procedure: Coconut oil (35 mg) and Lecithin (20 mg) are dissolved in 10 mL of ethanol by stirring at room temperature. To this solution, metalloporphyrin (20 mg) is added and heated to 40° C. while stirring to obtain a clear solution. Separately, Poloxamer-188 (20 mg) and Sodium Alginate (4.5 mg) are dissolved in 8 mL of distilled water and then added to the above ethanol solution while stirring at room temperature. Calcium chloride (0.5 mg) is dissolved in 1 mL of water and then added to the above mixture solution stirring at room temperature. The solvents are removed from this homogeneous solution under vacuum using a rotary evaporator. The residue is reconstituted with 10 mL of water by sonication for 20 min. The solution is frozen and lyophilized to obtain the final Metal porphyrin complex/alginate microparticles.

### EXAMPLE 10

### Preparation of Metalloporphyrin/Sodium Trimetaphosphate (STMP) Microparticles

Formulation contents (w/w): Coconut oil (35%), Lecithin (20%), metalloporphyrin (20%), Poloxamer-188 (20%) and Sodium trimetaphosphate (STMP) (5%)

Preparation procedure: Coconut oil (35 mg) and Lecithin (20 mg) are dissolved in 10 mL of ethanol by stirring at room temperature. To this solution, metalloporphyrin (20 mg) is added and heated to 40° C. while stirring to obtain a clear solution. Separately, Poloxamer-188 (20 mg) and STMP (5 mg) are dissolved in 8 mL of distilled water and then added to the above ethanol solution while stirring at room temperature. The solvents are removed from this homogeneous solution under vacuum using a rotary evaporator. The residue is reconstituted with 10 mL of water by sonication for 20 min. The solution is frozen and lyophilized to obtain the final metalloporphyrin /STMP microparticles.

### EXAMPLE 11

### Preparation of Metalloporphyrin Microparticle Formulations

Formulation contents (w/w): DSPC (45%), DPPC (45%), metalloporphyrin (10%).

Preparation procedure: metalloporphyrin (10 mg) is dissolved with sonication in 10 mL of 1 M ammonium hydroxide solution. DPPC (45 mg) dissolved in 45 mL ethanol is added to it. DSPC (45 mg) dissolved in 45 mL of ethanol is added and mixed well. The solution mixture is spray dried using Buchi-290 mini spray dryer. The set parameters are inlet temperature 55° C., outlet temperature 50° C., aspirator at 75%, solution feed pump at 8% and nitrogen flow at 30 mm. The feeding solution container and spray dryer compartments are protected from light during the process. The dry powder is stored frozen protected from light.

### EQUIVALENTS

Those skilled in the art will recognize, or be able to ascertain using no more than routine experimentation, many equivalents to the specific embodiments and methods described herein. Such equivalents are intended to be encompassed by the scope of the present invention.

All patents, patent applications, and literature references cited herein are hereby expressly incorporated by reference.

1. A microparticle comprising or consisting essentially of:
a metal porphyrin complex and a pharmaceutically acceptable stabilizer, provided the metal porphyrin complex is not zinc protoporphyrin, wherein the concentration of the metal porphyrin complex is from about 5% by weight of the microparticle.

2. The microparticle of claim 1, wherein the metal porphyrin complex is a metal mesoporphyrin complex or a metal protoporphyrin complex.

3. The microparticle of claim 1, wherein the metal is a neutral or ionic atom of an element selected from iron, tungsten, cobalt, magnesium, palladium, platinum, and chromium.

4. The microparticle of claim 1, wherein the metal is a neutral or ionic atom of tin.

5. The microparticle of claim 1, wherein the metal is neutral or ionic zinc; and the metal porphyrin complex is a metal mesoporphyrin complex.

6. The microparticle of claim 1, wherein the microparticle does not comprise an albumin.

7. The microparticle of claim 1, wherein the concentration of the metal porphyrin complex is from about 5% to about 30% by weight of the microparticle.

8. The microparticle of claim 1, wherein the metal is neutral or ionic iron.

9. The microparticle of claim 1, wherein the stabilizer comprises or consists essentially of a lipid.

10. The microparticle of claim 1, wherein the stabilizer comprises or consists essentially of a cationic lipid.

11. A composition comprising a plurality of microparticles of claim 1 and, optionally, a pharmaceutically acceptable carrier.

12. The composition of claim 11, wherein the composition does not comprise an albumin.

13. A method of inhibiting the activity of inducible heme oxygenase (HO-1) in a subject in need thereof, comprising:
administering to the subject an effective amount of a composition of claim 11.

14. A method of treating hepatic injury in a subject in need thereof, comprising:
administering to the subject an effective amount of a composition of claim 11.

15. The method of claim 14, wherein the hepatic injury is nonalcoholic steatohepatitis (NASH).

16. The method of claim 14, wherein the metal porphyrin complex is iron protoporphyrin (FePP).

17. A method of treating a cardiovascular disease such as myocardial ischemia-reperfusion injury, hypertension, or atherosclerosis, including endothelial dysfunction, inflammation, smooth muscle cell proliferation, and vasodilation; diabetes or obesity; hypoxia or ischemia; corneal inflammation; acute kidney injury; hormone dysregulation or imbalance; an infectious disease; cancer; aging, Parkinson's disease, or Alzheimer's disease; or brain hemorrhage such as subarachnoid hemorrhage, intracerebral hemorrhage, or stroke, in a subject in need thereof, comprising:
administering to the subject an effective amount of a composition of claim 11.

18. The method of claim 17, wherein the metal porphyrin complex is FePP.

19. A method of treating a porphyria or preventing an acute attack of a porphyria in a subject in need thereof, comprising:
administering to the subject an effective amount of a composition of claim 11.

20. The method of claim 19, wherein the metal porphyrin complex is SnPP or SnMP.

21. The method of claim 19, wherein the metal porphyrin complex is FePP or FeMP.

22. The method of claim 19, wherein the metal porphyrin complex is a heme.

23. The method of claim 19, wherein the metal porphyrin complex is hemin.

24. The method of claim 19, wherein the porphyria is AIP, HCP, VP, or ALAD.

25. The method of claim 19, wherein the subject is female.

26. The method of claim 19, wherein the subject is about 15 to about 45 years of age.

27. The method of claim 19, wherein the method is a method of preventing an acute attack of a porphyria; the subject is female and about 15 to about 45 years of age; and the composition is administered once a month.

28. The method of claim 19, wherein the method is a method of preventing an acute attack of a porphyria; the subject is female and about 15 to about 45 years of age; and the composition is administered about 8 days, about 7 days, about 6 days, about 5 days, about 4 days, about 3 days, about 2 days, or about 1 day prior to menstruation.

29. The method of claim 19, wherein the method is a method of treating a porphyria; the subject is female and about 15 to about 45 years of age; and
the composition is administered once a month.

30. The method of claim 19, wherein the method is a method of treating a porphyria; the subject is female and about 15 to about 45 years of age; and
the composition is administered about 8 days, about 7 days, about 6 days, about 5 days, about 4 days, about 3 days, about 2 days, or about 1 day prior to menstruation.

31. The method of claim 19, wherein the composition is administered orally.
